(19) United States
(12) Patent Application Publication
(10) Pub. No.: US 2026/0218254 A1
(43) Pub. Date: Jul. 30, 2026

(54) A PROCESS FOR PREPARING L-GLUFOSINATE FROM CYANHYDRINE OR CYANHYDRINE DERIVATIVES

(71) Applicant: BASF SE, Ludwigshafen (DE)

(72) Inventors: Gunther Zimmermann, Ludwigshafen (DE); Moritz Stefan Pott, Ludwigshafen (DE); Michael Breuer, Ludwigshafen (DE)

(21) Appl. No.: 19/159,016

(22) PCT Filed: Feb. 21, 2024
(86) PCT No.: PCT/EP2024/054394

§ 371 Date: Aug. 22, 2025

(30) Foreign Application Priority Data
Feb. 23, 2023 (EP) 23158204.0

(51) Int. Cl. C12P 13/04; A01N 57/20; A01P 13/02; C12N 9/80; C12N 9/86

(52) U.S. Cl. CPC C12P 13/04; A01N 57/20; A01P 13/02; C12N 9/80; C12N 9/86; C12Y 305/01; C12Y 305/02

(57) ABSTRACT

A process for preparing L-glufosinate and/or a salt thereof or an L-glufosinate alkyl ester and/or a salt thereof, wherein the L-glufosinate or the L-glufosinate alkyl ester have a molecular structure according to formula (I) wherein R¹ is H or C₁-C₈ alkyl, wherein the process comprises the step of reacting the following components in at least one reaction step: (1) a cyanhydrine or cyanhydrine derivative according to formula (II) wherein R¹ is H or C₁-C₈ alkyl, and R² is H, C₁-C₈ alkyl, C₆-C₁₀ aryl, C₇-C₁₀ aralkyl, C₄-C₁₀ cycloalkyl, or C₁-C₁₀ acyl; (2) a source of ammonia; (3) a source of carbon dioxide; and (4) at least two enzymes.

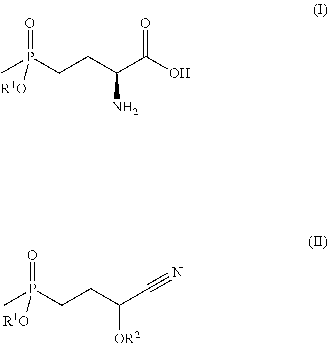

### TECHNICAL FIELD

The present invention relates to the preparation of L-glufosinate, in particular to the preparation of L-glufosinate from cyanhydrine or cyanhydrine derivatives.

### BACKGROUND

U.S. Pat. No. 4,168,963 describes various phosphorus-containing herbicidally active compounds, of which phosphinothricin (2-amino-4-[hydroxy(methyl)phosphinoyl]-butanoic acid: glufosinate) or its salts have attained commercial importance in the field of agrochemistry (agricultural chemistry). This herbicide glufosinate is a non-selective, foliarly-applied herbicide considered to be one of the safest herbicides from a toxicological or environmental standpoint.

Methods for the preparation of intermediates for the synthesis of such phosphorus-containing herbicidal active compounds, in particular glufosinate, are described for example in U.S. Pat. Nos. 4,521,348, 4,599,207 and 6,359,162.

WO 2015/173146 A1 describes the preparation of glufosinate starting from n-butyl (3-cyano-3-hydroxypropyl)methylphosphinat (ACM-H). Likewise, WO 2017/037012 A1 describes the preparation of glufosinate starting from n-butyl (3-cyano-3-acetoxypropyl)methylphosphinat (ACM).

### SUMMARY OF THE INVENTION

Phosphorus-containing cyanohydrins are valuable intermediates in various fields, in particular for the production of biologically active substances which can be used in the pharmaceutical or agrochemical sector. ACM and ACM-H are easily accessible from bulk chemicals and known intermediates in the synthesis of racemic glufosinate. However, there is no existing technology for the direct synthesis of L-Glufosinate from these intermediates. Hence, also the processes described above have the disadvantage that the glufosinate produced therefrom does not show any enantiomeric excess, in particular not in view of L-glufosinate.

Other current commercial chemical synthesis methods for glufosinate yield also only a racemic mixture of L- and D-glufosinate (Duke et al. 2010 Toxins 2:1943-1962). However, it is known that L-glufosinate is more potent than D-glufosinate (Ruhland et al. (2002) Environ. Biosafety Res. 1:29-37) in view of an herbicidal effect.

Thus, in view of the prior art listed above, it is an object of the present invention to provide a mild process for preparing L-glufosinate.

It is a further object of the present invention to provide a safe process for preparing L-glufosinate.

Moreover, it is an object of the present invention to provide a process for preparing L-glufosinate in an enantiomeric excess.

Finally, it is an object of the present invention to provide a process for preparing L-glufosinate from easily accessible source material.

It has surprisingly been found that at least one of the above objects can be achieved by the herein described process.

Hence, in a first aspect, the present invention relates to a process for preparing L-glufosinate and/or a salt thereof or an L-glufosinate alkyl ester and/or a salt thereof, wherein the L-glufosinate or the L-glufosinate alkyl ester have a molecular structure according to formula (I):

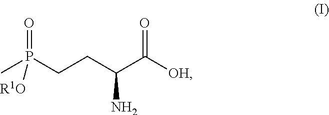

wherein R¹ is H or C₁-C₈ alkyl,
wherein the process comprises the step of reacting the following components in at least one reaction step:
(1) a cyanhydrine or cyanhydrine derivative according to formula (II)

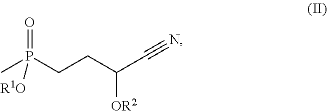

wherein
R¹ is H or C₁-C₈ alkyl, and R² is H, C₁-C₈ alkyl, C₆-C₁₀ aryl, C₇-C₁₀ aralkyl, C₄-C₁₀ cycloalkyl, or C₁-C₁₀ acyl;
(2) a source of ammonia;
(3) a source of carbon dioxide; and
(4) at least two enzymes.

In the following, preferred embodiments of the components of the process for preparing are described in further detail. It is to be understood that each preferred embodiment is relevant on its own as well as in combination with other preferred embodiments.

In a first preferred embodiment A1 of the first aspect of the present invention, the component (4) comprises, preferably consists of, (4a) at least one amidohydrolase acting on cyclic amides (EC 3.5.2) and (4b) at least one L-amidohydrolase acting on linear amides (EC 3.5.1).

In a second preferred embodiment A2 of the first aspect of the present invention, the components (1), (2), and (3) are initially contacted, subsequently component (4) is added, preferably first component (4a) and finally component (4b) are added.

In a third preferred embodiment A3 of the first aspect of the present invention, the components (1) to (4) are added in the same reaction step, preferably the reaction is carried out as a one pot reaction.

In a fourth preferred embodiment A4 of the first aspect of the present invention, the cyanhydrine is prepared by the reaction of an aldehyde and cyanide, preferably hydrogen cyanide or potassium cyanide, and wherein the aldehyde has a molecular structure according to formula (III):

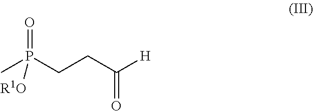

wherein R¹ is H or C₁-C₈ alkyl, preferably C₁-C₆ alkyl or H more preferably C₂-C₄ alkyl or H even more preferably ethyl or butyl or H, and most preferably ethyl.

In a fifth preferred embodiment A5 of the first aspect of the present invention, R¹ of formulae (I), (II), and/or (III) is H or C₁-C₈ alkyl, preferably H or C₁-C₆ alkyl, more preferably H or C₂-C₄ alkyl, even more preferably H, ethyl or butyl, and most preferably ethyl.

In a sixth preferred embodiment A6 of the first aspect of the present invention, the source of ammonia is selected from the list consisting of gaseous ammonia, solubilized ammonia, an ammonium salt, or mixtures thereof.

In a seventh preferred embodiment A7 of the first aspect of the present invention, the source of carbon dioxide is gaseous carbon dioxide, solubilized carbon dioxide, a carbonate salt, or mixtures thereof.

In an eighth preferred embodiment A8 of the first aspect of the present invention, the cyanhydrine or cyanhydrine derivative is a cyanhydrine derivative according to formula (IV):

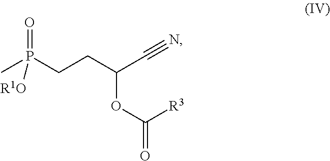

wherein R³ is C₁-C₈ alkyl, preferably C₁-C₄ alkyl, more preferably C₁-C₃ alkyl, and most preferably methyl.

In a ninth preferred embodiment A9 of the first aspect of the present invention, the amidohydrolase acting on cyclic amides (EC 3.5.2) is an L-amidohydrolase acting on cyclic amides (EC 3.5.2).

The invention further relates in a second aspect to a composition comprising a cyanhydrine or cyanhydrine derivative according to the formula (II)

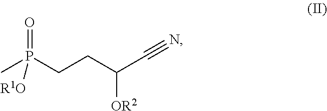

wherein
R¹ is H or C₁-C₈ alkyl, and
R² is H, C₁-C₈ alkyl, C₅-C₁₀ aryl, C₇-C₁₀ aralkyl, C₄-C₁₀ cycloalkyl, or C₁-C₁₀ acyl, and L-glufosinate and/or salts thereof.

The invention further relates in a third aspect to a method for selectively controlling weeds in an area, preferably containing a crop of planted seeds or crops that are resistant to glufosinate, comprising:
applying an effective amount of a composition comprising L-glufosinate and/or salts thereof obtained by the process of the present invention at an enantiomeric proportion of at least 50%, preferably in an enantiomeric excess of greater than 70%, over D-glufosinate and/or salts thereof and more than 0.01 wt.-% to less than 10 wt.-%, based on the total amount of the composition, of a cyanhydrine or cyanhydrine derivative according to the formula (II)

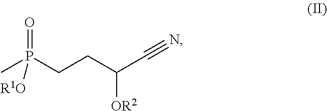

wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl,
to the area.

### DETAILED DESCRIPTION OF THE INVENTION

Before describing in detail exemplary embodiments of the present invention, definitions important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

The term “wt.-%” as used throughout herein stands for “percent by weight”.

The term “alkyl” as used herein denotes in each case a straight-chain or branched alkyl group having usually from 1 to 20 carbon atoms, preferably from 1 to 8 carbon atoms, frequently from 1 to 6 carbon atoms, more preferably 1 to 4 carbon atoms, e.g., 2 or 4 carbon atoms. Examples of alkyl groups are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, and n-hexyl.

Depending on the substitution pattern, the compounds according to the invention may have one or more stereocenters. Unless explicitly indicated otherwise (e.g., via a chemical formula) the invention preferably encompasses all stereoisomers, i.e. pure enantiomers, pure diastereomers, of the compounds according to the invention, and their mixtures, including racemic mixtures.

Preferred embodiments regarding the process for preparing L-glufosinate and/or a salt therefrom, or an L-glufosinate alkyl ester and/or the salt thereof are described in detail hereinafter. It is to be understood that the preferred embodiments of the invention are preferred alone or in combination with each other.

As indicated above, the present invention relates in one aspect to a process for preparing L-glufosinate and/or a salt thereof or an L-glufosinate alkyl ester and/or a salt thereof, wherein the L-glufosinate or the L-glufosinate alkyl ester have a molecular structure according to formula (I):

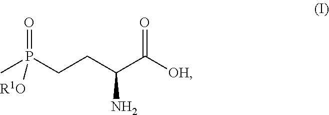

wherein R¹ is H or C₁-C₈ alkyl,
wherein the process comprises the step of reacting the following components in at least one reaction step:
(1) a cyanhydrine or cyanhydrine derivative according to formula (II)

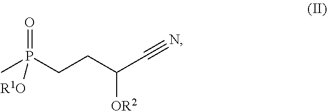

wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl;
(2) a source of ammonia;
(3) a source of carbon dioxide; and
(4) at least two enzymes.

Preferably, component (4) comprises, preferably consists of (4a) at least one amidohydrolase acting on cyclic amides (EC 3.5.2) and (4b) at least one L-Amidohydrolase acting on linear amides (EC 3.5.1).

It is to be understood that the preparing L-glufosinate and/or a salt therefrom, or an L-glufosinate alkyl ester and/or the salt thereof encompasses all stereoisomers, suitable salts of the respective L-glufosinate or its alkyl ester. Preferably, the alkyl ester of the L-glufosinate denotes the alkyl-P-ester of L-glufosinate. Further, the respective zwitterions are encompassed by the formula (I). Suitable salts are exemplarily hydrochloric acid salt, ammonium salts, and isopropylammonium salts. In this connection, the compound of formula (I) in particular encompasses two stereocenters, wherein one stereocenter is located at the phosphor atom and one stereocenter is located at the alpha carbon atom. The compound of formula (I) in particular encompasses all stereoisomers derived from the stereocenter at the phosphor atom.

The cyanhydrine or cyanhydrine derivative can be obtained via any suitable preparation process. Suitable processes are described inter alia in U.S. Pat. No. 4,521,348 B1, DE 3047024, U.S. Pat. No. 4,599,207 B1 U.S. Pat. No. 6,359,162 B1, CN 102372739 A, and CN 102399240 A.

In a preferred embodiment the cyanhydrine is prepared by the reaction of an aldehyde and cyanide, preferably hydrogen cyanide or potassium cyanide, wherein the aldehyde has a molecular structure according to formula (III):

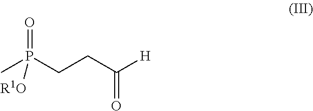

wherein R¹ is H or C₁-C₈alkyl, preferably H or C₁-C₆ alkyl, more preferably H or C₂-C₄ alkyl, even more preferably H, ethyl or butyl, and most preferably ethyl.

Such a reaction has the advantage that the cyanhydrine is prepared in situ and the solution can be directly used to carry out the process of the present invention.

Preferably, R¹ of formulae (II) and/or (III) is H or C₁-C₈ alkyl. Thus, preferably, the cyanhydrine or cyanhydrine derivative is a precursor for L-glufosinate or the C₁-C₈ protected alkyl ester of L-glufosinate, preferably the C₁-C₈ protected alkyl-P-ester of L-glufosinate.

Likewise, preferably, R² of formula (II) is H, C₁-C₈ alkyl, C₆-C₁₀ aryl, C₇-C₁₀ aralkyl, C₄-C₁₀ cycloalkyl, or C₁-C₁₀ acyl. More preferably, R² of formula (II) is H or C₁-C₁₀ acyl, and most preferably R² of formula (II) is H or acetyl.

Thus, in a first most preferred embodiment of the present invention, the cyanhydrine is n-butyl (3-cyano-3-hydroxypropyl)methylphosphinat (ACM-H)

Preferably cyanhydrine derivative is a cyanhydrine derivative according to formula (IV):

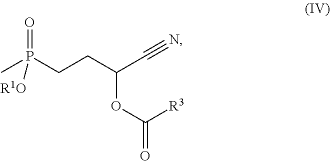

wherein R³ is C₁-C₈ alkyl, preferably C₁-C₄ alkyl, more preferably C₁-C₃ alkyl, and most preferably methyl.

Hence, in a second most preferred embodiment of the present invention, the cyanhydrine derivative is n-butyl (3-cyano-3-acetoxypropyl)methylphosphinat (ACM).

The process can be carried out in several steps. Preferably, in case the process is carried out in several steps, components (1) to (3) are initially added followed by component (4), preferably first component (4a) and finally component (4b). If the process is carried out in more than one step, it is preferably carried out in two steps, wherein the first step includes the reaction of components (1) to (3), whereas the second step includes the addition of component (4), preferably (4a) and (4b). In another preferred embodiment, the process is carried out in one step, i.e., is a one-pot process.

In case the process is carried out in two steps, it is preferred that the first step is carried out at higher temperatures than the second step. Preferably, the first step is carried out at a temperature in the range of from 40 to 100° C., more preferably 50 to 90° C.; and most preferably 75 to 85° C. It has been surprisingly found that with higher temperatures in the first step, the overall conversion rates are increased. Preferably, the first step is carried out for at least half an hour, more preferably for at least one hour. Usually, the first step is finished after 1 hour.

In case the process is carried out in two steps, the second step is preferably carried out at temperatures suitable for the enzymes according to component (4), preferably (4a) and (4b), whereby suitable means that first the enzymes are stable within this temperature range and second the reaction catalyzed by these enzymes takes place, preferably at an optimal conversion rate. Preferably, the temperature of the second step is as applied for the one pot reaction as set out below.

In case the process is carried out in one step, i.e., is a one-pot process, it is carried out at a temperature in the range of 20 to 50° C., preferably in the range of 25 to 45° C., more preferably in the range of 30 to 42° C., and most preferably in the range of 32 to 40° C.

In a preferred embodiment of the present invention, the reaction is performed at a pH of 6 to 11, preferably of 6.5 to 10, more preferably of 7 to 9.5 and in particular of 7.5 to 9. The pH can be adjusted using alkali hydroxide, more preferably sodium hydroxide or potassium hydroxide, and in particular potassium hydroxide. However, most preferably, the pH is adjusted by the source of ammonia and/or source of carbon dioxide. In particular, the pH can be controlled by the amount of ammonium salt or carbonate salt, preferably ammonium carbonate or ammonium hydrogen carbonate, added to the solution or by the amount of gaseous ammonia and/or carbon dioxide lead through the solution or by both at the same time.

In a preferred embodiment of the present invention, the reaction is performed under aqueous conditions, preferably in degassed aqueous phosphate buffer, more preferably degassed aqueous potassium phosphate buffer. However, also the buffer effect of the solution can be adjusted by the source of ammonia and/or source of carbon dioxide. In particular, the pH can be controlled by the amount of ammonium salt or carbonate salt, preferably ammonium carbonate or ammonium hydrogen carbonate, added to the solution or by the amount of gaseous ammonia and/or carbon dioxide lead through the solution or by both at the same time.

In a preferred embodiment of the present invention, the process is carried out under stirring, preferably at 50 to 1000 rpm, more preferably at 100 to 800 rpm, even more preferably at 150 to 600 rpm, still more preferably at 180 to 400 rpm, and most preferably at 200 to 300 rpm.

Preferably, in the process of the present invention, any suitable amidohydrolase acting on cyclic amides (EC 3.5.2) may be used.

Such amidohydrolase acting on cyclic amides (EC 3.5.2) that can be used in the process of the invention include those from Defluviimonas alba, Rhodococcus erythropolis, Streptomyces coelicolor, Brevibacillus agri, Paenarthrobacter aurescens, Arthrobacter crystallopoietes, Bacillus sp. TS-23, Bacillus fordii, Jannaschia sp., Pseudomonas putida, Geobacillus stearothermophilus, Thermus sp., Dictyostelium discoideum, Rhizobium meliloti, Pseudomonas aeruginosa, Rhizobium radiobacter, Pseudomonas fluorescens, Glycine max, Robinia pseudoacacia, Bacillus licheniformis, Aedes aegypti, Agrobacterium fabrum, Arthrobacter sp., and the like, preferably Defluviimonas alba.

Suitable amidohydrolase acting on cyclic amides (EC 3.5.2) may be selected from the group consisting of Q8RSQ2 and variants thereof, 069809 and variants thereof, Q846U5_9BACL and variants thereof, P81006 and variants thereof, Q84FR6_9MICC and variants thereof, Q56S49_9BACI and variants thereof, A1E351_9BAC and variants thereof, Q28SA7 and variants thereof, Q59699 and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B5L363 and variants thereof, 11MEH3 and variants thereof, Q6S4R9 and variants thereof, Q65LN0 and variants thereof, Q171F8 and variants thereof, Q8U8Z6 and variants thereof, P42084 and variants thereof, Q88NW7 and variants thereof, P25995 and variants thereof, Q3Z354 and variants thereof, B1XEG2 and variants thereof, Q9F465_PAEAU and variants thereof, Q01262.1 and variants thereof, A0A250DXG4_GEOSE and variants thereof, A1SPN2 and variants thereof, Q9WYH0 and variants thereof, P58329 and variants thereof, A1SGT4 and variants thereof, E3JD18 and variants thereof, HUTI_BDEBA and variants thereof, A0A161KD37_9CHLR and variants thereof, 10GL27_CALEA and variants thereof, A0A068WGW0_ECHGR and variants thereof, A0A1J4XHR4_9BACT and variants thereof, A0A1C₄QIY5_9ACTN and variants thereof, A0A0K2UMP4_LEPSM and variants thereof, A0A0F5Q0A2_9RHIZ and variants thereof, A0A024KHS5_9RHIZ and variants thereof, A0A060UM69_9PROT and variants thereof, A3DKS9_STAMF and variants thereof, W2EWT0_9ACTN and variants thereof, A0A0B1T9I4_OESDE and variants thereof, A0A0A7 LM60_9BACT and variants thereof, A0A087M7T5_9RHIZ and variants thereof, COC180_9FIRM and variants thereof, A0A159Z531_9RHOB and variants thereof, R5JTP2_9CLOT and variants thereof, A0A010RM85_9PEZI and variants thereof, E1R8C9_SEDSS and variants thereof, A0A010YEH8_9BACT and variants thereof, A0A031LV69_9CREN and variants thereof, A0A1F9 QT17_9BACT and variants thereof, ALLB_BACVZ and variants thereof, HUTI_FLAPJ and variants thereof, A0A073J5J1_9BACT and variants thereof, A0A034W2Q8_BACDO and variants thereof, A0A0D8IVV8_9FIRM and variants thereof, A0A0B5QKE4_CLOBE and variants thereof, A0A098B7X6_DESHA and variants thereof, A0A0B5H4M8_9EURY and variants thereof, A0A0C1YDP1_9ACTN and variants thereof, Q981H2_RHILO and variants thereof, T1EEH7_HELRO and variants thereof, A0A060DTG8_AZOBR and variants thereof, A0A011MGZ5_MANHA and variants thereof, A0A060LYB6_9BACI and variants thereof, SOF3L7_CHOCR and variants thereof, A0A133VNR0_9EURY and variants thereof, A0A133U7U9_9EURY and variants thereof, A0A0U2XD52_ECOLX and variants thereof, M1YZY6_NITG3 and variants thereof, TON9X6_9EURY and variants thereof, TOLMU2_9EURY and variants thereof, A0A0N1GBZ8_9ACTN and variants thereof, HUTI_ANASK and variants thereof, A0A031JUP0_9SPHN and variants thereof, A0A061N9L2_9BACL and variants thereof, A0A017T4D2_9DELT and variants thereof, A0A174ADZ3_9FIRM and variants thereof, A0A021X7D5_9RHIZ and variants thereof, A0A021XAC5_9RHIZ and variants thereof, A0A0C2UIW0_9BACL and variants thereof, A0A1F8NGY1_9CHLR and variants thereof, D3F1S3_CONWI and variants thereof, A0A021XG06_9RHIZ and variants thereof, U7V9Q6_9FUSO and variants thereof, D6XY37_BACIE and variants thereof, A0A0J1FAI4_9FIRM and variants thereof, B5Y9A6_COPPD and variants thereof, PHYDA_ECOK1 and variants thereof, A0A0A9X9B7_LYGHE and variants thereof, A0A0S8H576_9BACT and variants thereof, A0A151ABI4_9EURY and variants thereof, A0A064AFD7_9FUSO and variants thereof, A0A0C₂FCG7_9ACTN and variants thereof, A0A0S8CI48_9CHLR and variants thereof, A0A1F9CZ74_9DELT and variants thereof, A0A0A3YKD1_9ENTR and variants thereof, A0A084R4T2_STACH and variants thereof, A0A070A1Z0_9PROT and variants thereof, A0A1J4J4Y8_9EUKA and variants thereof, R1BR72_EMIHU and variants thereof, RIDD72_EMIHU and variants thereof, AOA1L0FIA0_9ASCO and variants thereof, F7DRE9_ORNAN and variants thereof, AOLK75_SYNFM and variants thereof, A0A0Q1A918_9BACT and variants thereof, H2YZ10_CIOSA and variants thereof, 14 YD99_WALMC and variants thereof, A0A077YYH5_TRITR and variants thereof, A0A077Y189_9SPHI and variants thereof, A0A089K5P4_9BACL and variants thereof, A0A0Q7W2T1_9RHIZ and variants thereof, A0A174NIK6_9FIRM and variants thereof, A0A0D5NFS5_9BACL and variants thereof, A0A0D5NNJ7_9BACL and variants thereof, A0A1H2AV66_9BACL and variants thereof, A0A0Q4RXY0_9BACL and variants thereof, A0A0Q7SB75_9BACL and variants thereof, A0A015NM92_9BACL and variants thereof, AOA100VRN2_PAEAM and variants thereof, W4BDJ0_9BACL and variants thereof, A0A147K2G0_9EURY and variants thereof, A0A0W8FVM4_9ZZZZ and variants thereof, A0A147JXR0_9EURY and variants thereof, E8R8J7_DESMO and variants thereof, D5U113_THEAM and variants thereof, A0A1F8T9J2_9CHLR and variants thereof, G3C₉₅₂_9ARCH and variants thereof, Q6YNI0_9MICC and variants thereof, A0A1G0YIQ9_9BACT and variants thereof, A0A1J5EHQ6_9DELT and variants thereof, A0A1J5E082_9DELT and variants thereof, A0A1C₄PKD1_9ACTN and variants thereof, H8GX25_DEIGI and variants thereof, A0A1H5ZFN3_9BACT and variants thereof, A0A0M9Z5S1_9ACTN and variants thereof, A0A1B2HNC5_9PSEU and variants thereof, A0A1B2GNI8_STRNR and variants thereof, A0A1F8LBZ3_9CHLR and variants thereof, A0A1F8NMM2_9CHLR and variants thereof, A0A1F8SDV1_9CHLR and variants thereof, A0A1H1PLX0_9BACT and variants thereof, 10IDC5_PHYMF and variants thereof, A0A0Q518X4_9DEIO and variants thereof, A0A0F4JEH6_9ACTN and variants thereof, BAD75708.1, *WP_014453859.1 and variants thereof, *WP_046170519.1 and variants thereof, *CDP53201.1 and variants thereof, *WP_035078314.1 and variants thereof, *WP_042803791.1 and variants thereof, *EQB70510.1 and variants thereof, *EQB65904.1 and variants thereof, *WP_023512514.1 and variants thereof, *WP_023514195.1 and variants thereof, *WP_023516147.1 and variants thereof, *KGT87257.1 and variants thereof, *WP_045756097.1 and variants thereof, *WP_056239694.1 and variants thereof, *KUO41395.1 and variants thereof, *KOV34818.1 and variants thereof, *ANZ15483.1 and variants thereof, *KJY32595.1 and variants thereof, and mixtures thereof, wherein variants are defined as polypeptide sequences with at least 80%, preferably 90%, and most preferably 95%, sequence identity to the respective polypeptide sequence.

Further preferably, the amidohydrolase acting on cyclic amides (EC 3.5.2) is selected from the group consisting of O69809 and variants thereof, Q846U5_9BACL and variants thereof, P81006 and variants thereof, Q84FR6_9MICC and variants thereof, Q56S49_9BACI and variants thereof, A1E351_9BACI and variants thereof, Q28SA7 and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B5L363 and variants thereof, P42084 and variants thereof, P25995 and variants thereof, Q3Z354 and variants thereof, B1XEG2 and variants thereof, Q9F465_PAEAU and variants thereof, A0A161KD37_9CHLR and variants thereof, A0A1J4XHR4_9BACT and variants thereof, A0A1C₄QIY5_9ACTN and variants thereof, A0A0K2UMP4_LEPSM and variants thereof, A0A159Z531_9RHOB and variants thereof, E1R8C₉_SEDSS and variants thereof, A0A1F9 QT17_9BACT and variants thereof, A0A0D8IVV8_9FIRM and variants thereof, A0A0B5QKE4_CLOBE and variants thereof, A0A0N1GBZ8_9ACTN and variants thereof, A0A174ADZ3_9FIRM and variants thereof, U7V9Q6_9FUSO and variants thereof, A0A0J1FAI4_9FIRM and variants thereof, PHYDA_ECOK1 and variants thereof, A0A0S8H576_9BACT and variants thereof, A0A1J4J4Y8_9EUKA and variants thereof, A0A0D5NFS5_9BACL and variants thereof, A0A0D5NNJ7_9BACL and variants thereof, A0A1H2AV66_9BACL and variants thereof, A0A0Q4RXY0_9BACL and variants thereof, A0A0Q7SB75_9BACL and variants thereof, AOA100VRN2_PAEAM and variants thereof, W4BDJ0_9BACL and variants thereof, A0A1J5E082_9DELT and variants thereof, A0A1H5ZFN3_9BACT and variants thereof, A0A1F8NMM2_9CHLR and variants thereof, A0A1F8SDV1_9CHLR and variants thereof, A0A1H1PLX0_9BACT and variants thereof, A0A0Q518X4_9DEIO and variants thereof, *WP_046170519.1 and variants thereof, *WP_023514195.1 and variants thereof, *WP_023516147.1 and variants thereof, and *ANZ15483.1, and mixtures thereof, wherein variants are defined as polypeptide sequences with at least 80%, preferably 90%, and most preferably 95%, sequence identity to the respective polypeptide sequence.

Further, suitable amidohydrolases acting on cyclic amides (EC 3.5.2) may be selected from the group consisting of, Q846U5_9BACL and variants thereof, P81006 and variants thereof, Q84FR6_9MICC and variants thereof, Q56S49_9BACI and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B1XEG2 and variants thereof, A0A161KD37_9CHLR and variants thereof, A0A159Z531_9RHOB and variants thereof, E1R8C₉_SEDSS and variants thereof, A0A1F9 QT17_9BACT and variants thereof, A0A0B5QKE4_CLOBE and variants thereof, A0A0N1GBZ8_9ACTN and variants thereof, BAD75708.1 and variants thereof, A0A064AFD7_9FUSO and variants thereof, and mixtures thereof, wherein variants are defined as polypeptide sequences with at least 80%, preferably 90%, and most preferably 95%, sequence identity to the respective polypeptide sequence.

In a preferred embodiment, the amidohydrolase acting on cyclic amides (EC 3.5.2) is selected from the group consisting to Q846U5_9BACL and variants thereof, P81006 and variants thereof, Q84FR6_9MICC and variants thereof, A0A399DRQ3_9DEIN and variants thereof, B1XEG2 and variants thereof, A0A161KD37_9CHLR and variants thereof, A0A159Z531_9RHOB and variants thereof, E1R8C₉_SEDSS and variants thereof, A0A1F9 QT17_9BACT and variants thereof, A0A0B5QKE4_CLOBE and variants thereof, A0A0N1GBZ8_9ACTN and variants thereof, BAD75708.1 and variants thereof, A0A064AFD7_9FUSO, and mixtures thereof, wherein variants are defined as polypeptide sequences with at least 80%, preferably 90%, and most preferably 95%, sequence identity to the respective polypeptide sequence.

Most preferably, the amidohydrolase acting on cyclic amides (EC 3.5.2) is selected from the group consisting of Q45515, Q44184 and variants thereof, A0A1C₄QIY5_9ACTN and variants thereof, A0A0K2UMP4_LEPSM and variants thereof, *WP_046170519.1 and variants thereof, and E1R8C₉_SEDSS and variants thereof, A0A159Z531_9RHOB and variants thereof, and mixtures thereof, wherein variants are defined as polypeptide sequences with at least 80%, preferably 90%, and most preferably 95%, sequence identity to the respective polypeptide sequence.

It is to be understood that the above outlined amidohydrolase acting on cyclic amides (EC 3.5.2) are indicated in the nomenclature of the database identifier according to the Uniprot (www.uniprot.org). or the NCBI protein database (www.ncbi.nlm.nih.gov/protein), where sequences from NCBI are indicated by an “*” at the beginning of the respective database identifier.

In a preferred embodiment of the present invention, the amidohydrolase acting on cyclic amides (EC 3.5.2) is an L-amidohydrolase acting on cyclic amides (EC 3.5.2).

In a preferred embodiment of the present invention, R1 in formulae (I), (II), (III), and (IV) is H or C₁-C₆ alkyl, preferably H or C₂-C₄ alkyl, more preferably H, ethyl or butyl, and most preferably ethyl.

Suitable L-Amidohydrolases acting on linear amides (EC 3.5.1) are preferably selected from the group consisting of EC 3.5.1 Hydrolases acting on linear amides, EC 3.5.1.87 N-carbamoyl-L-amino-acid hydrolase, 3.5.1.77 N-carbamoyl-D-amino-acid hydrolase, and mixtures thereof. Suitable L-Amidohydrolases acting on linear amides (EC 3.5.1) that can be used in the process include those selected from the group consisting of A0A7Y0T4N7_9RHIZ and variants thereof, Q88FQ3_PSEPK and variants thereof, Q88Q81_PSEPK and variants thereof, A0A126S6J4_PSEPU and variants thereof, Q8VUL6_9PSED and variants thereof, H9B8T5_9PSED and variants thereof, Q9FB05_9PSED and variants thereof, COZCM8_BREBN and variants thereof, COZ7R⁵_BREB and variants thereof, A0A0K9YX84_9BACL and variants thereof, E3HUL6_ACHXA and variants thereof, A0A1V9BSS3_9BACI and variants thereof, A0A1V9BSS3_9BACI and variants thereof, Q9F464 and variants thereof, A0A4D7Q548_GEOKU and variants thereof, Q9F464 and variants thereof, A0A2S9D976_9MICC and variants thereof, A0A116VZZ4_9RHIZ and variants thereof, A0A1L6RE91_9LACT and variants thereof, A0A3E0C₉₉₆_9BURK and variants thereof, A0A3M7BGJ4_HORWE and variants thereof, A0A2D7YQN7_9GAMM and variants thereof, A0A535Y1H2_UNCCH and variants thereof, A0A223E415_9BACI and variants thereof, M2VSE9_GALSU and variants thereof, A0A3T0K6C0_9GAMM and variants thereof, A0A416FGE1_9CLOT and variants thereof, D1P143_9GAMM and variants thereof, A0A6P2ISL4_BURL3 and variants thereof, A0A3S6Z2M9_9FIRM and variants thereof, A0A0C1US49_9BACT and variants thereof, A0A1Y4GC62_9BACT and variants thereof, A0A3D3VMN7_9BACT and variants thereof, A0A2K8L549_9PROT and variants thereof, A0A1G0MC89_9BACT and variants thereof, A0A1M6WYS1_SELRU and variants thereof, A0A2K2BY13_POPTR and variants thereof, A0A510DYR5_9CREN and variants thereof, A0A5Y3XFN7_SALER and variants thereof, A0A3811B54_CLODI and variants thereof, A0A2V3IQW6_9FLOR and variants thereof, and mixtures thereof, wherein variants are defined as polypeptide sequences with at least 80%, preferably 90%, and most preferably 95%, sequence identity to the respective polypeptide sequence. Most preferably the L-Amidohydrolase acting on linear amides (EC 3.5.1) is selected from the group consisting of A0A3E0C₉₉₆_9BURK and variants thereof, A0A535Y1H2_UNCCH ( ) and variants thereof, A0A6P2ISL4_BURL3 ( ) and variants thereof, A0A1Y4GC62_9BACT (and variants thereof, wherein variants are defined as polypeptide sequences with at least 80%, preferably 90%, and most preferably 95%, sequence identity to the respective polypeptide sequence. It is to be understood that the above outlined L-Amidohydrolases acting on linear amides (EC 3.5.1) are indicated in the nomenclature of the database identifier according to the Uniprot database (www.uniprot.org).

In a preferred embodiment of the present invention, R¹ in formulae (I) is C₁-C₈ alkyl, preferably C₁-C₆ alkyl, more preferably C₂-C₄ alkyl, even more preferably ethyl or butyl, and most preferably ethyl, and the process of the present invention further comprises the step of deprotecting under acidic conditions. In this connection any suitable acid is possible. Preferably hydrochloric acid or sulfuric acid are being used.

In a preferred embodiment of the present invention, the process further comprises the addition of a Racemase enzyme. Any suitable Racemase enzyme may be possible. Suitable Racemase enzymes are selected from the group consisting of EC 5.1 Racemase, EC 5.1.1 Racemases acting on amino acids and derivatives, EC 5.1.99.5 racemase, and mixtures thereof. Suitable Racemase enzymes that can be used in the process include those selected from group consisting of Q9RYA6_DEIRA and variants thereof, Q9F466 and variants thereof, Q9F466 and variants thereof, A0A7L5BQP9_9RHIZ and variants thereof, Q00924 and variants thereof, F7X6X4_SINMM and variants thereof, A0A6V7ACK5_RHIRD and variants thereof, A0A7Y0XLH3_9RHIZ and variants thereof, A0A5B8XR30_9DELT and variants thereof, A0A533QH78_9PROT and variants thereof, A0A3M9Z0A0_9CYAN and variants thereof, A0A3A0A4T5_9CHLR and variants thereof, A0A1F6C9P8_HANXR and variants thereof, A0A4S0NM85_9RHIZ and variants thereof, A0A1V51086_9SPIR and variants thereof, AOA6P0NEY4_9CYAN and variants thereof, A0A2K0YBY8_9SPHN and variants thereof, A0A1H5NHN7_9RHIZ and variants thereof, A0A317KUZ3_9ACTN and variants thereof, A0A430VJ34_THESC and variants thereof, A0A1J5KHA5_9PROT and variants thereof, A0A535LIJ4_9CHLR and variants thereof, A0A2T6KHH4_9RHOB and variants thereof, A0A3G8JSD5_9ACTN and variants thereof, A0A3A9JRT3_9THEO and variants thereof, A0A2N7WBP6_9BURK and variants thereof, A0A1A2N8C₄_9MYCO and variants thereof, A0A1R3TB43_9RHIZ and variants thereof, X1T733_9ZZZZ and variants thereof, A0A6P1SX79_9RHOB and variants thereof, A0A0Q5VT22_9RHIZ and variants thereof, A0A2N1RKS5_9SPIR and variants thereof, A0A529XJR5_9RHIZ and variants thereof, A0A358TXS4_9FIRM and variants thereof, A0A1Q9UJX6_9ACTN and variants thereof, A0A434WJY9_9RHIZ and variants thereof, A0A4R7C3Y1_9RHIZ and variants thereof, A0A2T4IRF7_9RHIZ and variants thereof, A0A2E8B427_9PLAN and variants thereof, A0A538D678_9ACTN and variants thereof, A0A1W6Z0D5_9BORD and variants thereof, A0A3P1UKI1_9RHIZ and variants thereof, U2S1Q0_9FIRM and variants thereof, A0A3D5IHC5_AGRSP and variants thereof, A0A3D5JEU3_9DELT, and variants thereof, wherein variants are defined as polypeptide sequences with at least 80%, preferably 90%, and most preferably 95%, sequence identity to the respective polypeptide sequence, and mixtures thereof. It is to be understood that the above outlined Racemase enzymes are indicated in the nomenclature of the database identifier according to the Uniprot database (www.uniprot.org). Most preferably, the Racemase enzyme is selected from the group consisting of A0A6V7ACK5_RHIRD and variants thereof, A0A2T6KHH4_9RHOB and variants thereof, wherein variants are defined as polypeptide sequences with at least 80%, preferably 90%, and most preferably 95%, sequence identity to the respective polypeptide sequence.

In a preferred embodiment of the present invention, the process further comprises the addition of an N-Carbamoyl amino acid racemase enzyme. Any suitable N-Carbamoyl amino acid racemase enzyme may be possible.

In a preferred embodiment of the present invention, the process further comprises the addition of a Racemase enzyme as outlined above and an N-Carbamoyl amino acid racemase enzyme.

In a preferred embodiment of the present invention, all steps of the process are carried out in a single container. In this connection, all components are preferably substantially added at the start of the reaction.

In a preferred embodiment of the present invention, at least 15%, preferably at least 20%, more preferably at least 30%, even more preferably at least 50%, and in particular at least 70%, of the cyanhydrine or cyanhydrine derivative according to formula (II) is converted to L-glufosinate and/or a salt therefrom or the L-glufosinate alkyl ester and/or a salt therefrom.

In case the L-glufosinate alkyl ester or salt thereof has been produced, the process of the present invention can involve a final step of deprotecting the L-glufosinate alkyl ester or salt thereof to yield the L-glufosinate chloride thereof, which can be further transformed to L-glufosinate by increasing the pH value of the solution. A respective detailed description of this process step can be found in EP 0 508 296 A1.

Preferably, in the process according to the first aspect of the present invention, the L-glufosinate and/or the salt thereof or the L-glufosinate alkyl ester and/or the salt thereof are prepared in enantiomeric excess, preferably in an enantiomeric excess of more than 85%, more preferably more than 90%, even more preferably more than 95%, and most preferably more than 99%.

The applied enzymes may be applied via any suitable known in the art way. In a preferred embodiment of the present invention, the applied enzymes are applied as cleared cell lysate, whole cells, or immobilized enzymes.

Alternatively, some or all the components other than L-glufosinate can be removed from the biotransformation mixture, the mixture optionally concentrated, and then the mixture can be used directly (and/or with the addition of various adjuvants) for the prevention or control of weeds. The biotransformation mixture, in some instances, can be used directly (and/or with the addition of various adjuvants) for the prevention or control of weeds.

Additional steps to further purify the L-glufosinate can be added. Such further purification and isolation methods include ion exchange, extraction, salt formation, crystallization, and filtration; each may be used multiple times or in suitable combination. Enzymes can be removed by simple filtration if supported, or if free in solution by the use of ultrafiltration, the use of absorbents like celite, cellulose or carbon, or denaturation via various techniques known to those skilled in the art.

Ion exchange processes effect separation by selective adsorption of solutes onto resins chosen for this purpose. Because products and impurities must be dissolved in a single solution prior to adsorption, concentration of the purified product stream by evaporation or distillation prior to isolation is usually required. Examples of the use of ion exchange for purification are described by Schultz et al., and in EP0249188 (A2).

Purification may be achieved by the formation of an insoluble salt of L-glufosinate by the addition of a suitable acid, including hydrochloric acid, sulfuric acid, phosphoric acid, nitric acid, acetic acid, and the like. Similarly, the purification may be achieved by the addition of a suitable base to form an insoluble salt. Useful bases include hydroxides, carbonates, sulfates and phosphates of alkali metals or hydroxides, carbonates, sulfates, and phosphates of alkali earth metals. Other bases which contain nitrogen may be used, including ammonia, hydroxylamine, isopropylamine, triethylamine, tributylamine, pyridine, 2-picoline, 3-picoline, 4-picoline, 2,4-lutidine, 2,6-lutidine, morpholine, N-methymorpholine, 1,8-diazabicyclo[5.4.0]undec-7-ene, and dimethylethanolamine. It may be advantageous to concentrate the mixture or to add a solvent (or both) to maximize yield and optimize purity of the desired salt. Solvents suitable for this purpose include those in which the solubility of the desired salt is very low (such solvents are often called “anti-solvents”). Salts of L-glufosinate can be transformed into forms of glufosinate suitable for formulation by standard methods known to those skilled in the art. Alternatively, the L-glufosinate can be isolated as a zwitterion.

U.S. Pat. No. 9,255,115 B2 describes how the hydrochloric acid salt of L-glufosinate can be converted to the zwitterionic form with a base such as sodium hydroxide or sodium methoxide and then crystallized from aqueous alcohol solvent to afford L-glufosinate in relatively high purity. This method has the advantage of producing crystalline L-glufosinate that is not hygroscopic and therefore maintains a higher purity compared to amorphous L-glufosinate when exposed to humidity over time.

Other salts of L-glufosinate are known in the art. U.S. Pat. Nos. 5,767,309 and 5,869,668 teach the use of chiral alkaloid bases to form diastereomeric salts with racemic glufosinate. Purification is achieved because the salt of L-glufosinate precipitates from solution in much larger quantity than the corresponding salt of D-glufosinate. Therefore, this method could be used with the present invention to obtain L-glufosinate with high enantiomeric excess, if desired.

Optionally, purification may be achieved by first crystallizing one or more impurities, removing the impurities by filtration, and then further purifying L-glufosinate from the resulting filtrate by forming a salt as previously described. This is advantageous if unreacted amine donor can be partially or completely isolated and used in subsequent reactions. Similarly, unreacted cyanhydrine or cyanhydrine derivative according to formula (II) that is partially or completely isolated may be recycled for use in subsequent reactions.

Extraction may be used to purify the product. DE 3920570 C₂ describes a process in which excess glutamic acid (used as the amine donor) is precipitated by adjusting the solution pH to 3.7 to 4.2 with sulfuric acid. After filtering the glutamic acid, the filtrate pH is lowered to 1-2 whereupon other impurities are extracted into a solvent. After extraction and concentration, ammonia is added to the aqueous solution to a pH of 5-7 whereupon ammonium sulfate precipitates. The ammonium sulfate is removed by filtration and the resulting filtrate is concentrated to afford the ammonium salt of L-glufosinate.

Isolation of L-glufosinate or its salts may be desirable, for example, for the purpose of shipping solids to the location of formulation or use. Typical industrial methods of isolation may be used, for example, a filtration, centrifugation, etc. Isolated product often requires the removal of water, volatile impurities, and solvents (if present) and typical industrial drying equipment may be used for this purpose. Examples of such equipment include ovens, rotating drum dryers, agitated 17 PCT/EP2024/054394 WO 2024/175643 dryers, etc. In some cases, it may be advantageous to use a spray dryer.

It is not necessary to produce a solid product after purification. This may be advantageous if the formulation of L-glufosinate is to occur at the same site used for L-glufosinate production. L-glufosinate and many of its salts are readily soluble in water, and water is a convenient liquid to use for formulating products. For example, the amine donor is isolated by filtration and the resulting filtrate is concentrated by distillation. The pH of the filtrate may be adjusted to a desirable value and the resulting solution may be used as is or blended with formulation ingredients. In another example, a slurry of L-glufosinate or one of its salts may be prepared as described above and isolated by filtration. The solid could be dissolved directly on the filter by adding water or a suitable solvent to obtain a solution of L-glufosinate.

As mentioned above, the invention further relates in a second aspect to a composition comprising a cyanhydrine or cyanhydrine derivative according to the formula (II)

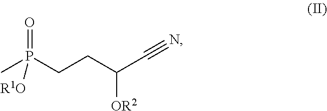

wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl, and L-glufosinate and/or salts thereof.

Suitable salts are hydrochloric acid salt, ammonium salts, and isopropylammonium salts. It is further to be understood that the respective zwitterion of L-glufosinate is also encompassed.

In a preferred embodiment of the present invention, the amount of L-glufosinate and/or salts thereof is at least 20 wt.-%, preferably at least 30 wt.-%, more preferably at least 40 wt.-%, even more preferably at least 50 wt.-%, still more preferably at least 60 wt.-%, and in particular at least 70 wt.-% or at least 80 wt.-%, based on the total amount of the cyanhydrine or cyanhydrine derivative according to the formula (II), and L-glufosinate and/or salts thereof.

In a preferred embodiment of the present invention, the amount of L-glufosinate and/or salts thereof is in the range of 20 to 99 wt.-%, preferably of 30 to 98 wt.-%, more preferably of 40 to 96 wt.-%, even more preferably of 50 to 95 wt.-%, still more preferably of 60 to 94 wt.-%, and in particular at least 70 to 90 wt.-% or at least 80 to 90 wt.-%, based on the total amount of the cyanhydrine or cyanhydrine derivative according to the formula (II), and L-glufosinate and/or salts thereof.

The composition can comprise the cyanhydrine or cyanhydrine derivative according to the formula (II) in an amount of up to 30 wt.-%, preferably up to 20 wt.-%, more preferably up to 10 wt.-%, even more preferably up to 5 wt.-%, still more preferably up to 2.5 wt.-%, and in particular up to 1 wt.-%, based on the total amount of the cyanhydrine or cyanhydrine derivative according to the formula (II), and L-glufosinate and/or salts thereof.

In one preferred embodiment of the present invention, the herein described composition may be used directly as an herbicidal composition or as an ingredient in a formulated herbicidal product.

The compositions described herein are useful for application to a field of crop plants for the prevention or control of weeds. The composition may be formulated as a liquid for spraying on a field. The glufosinate, preferably the L-glufosinate, is provided in the composition in effective amounts. As used herein, effective amount means from about 10 grams active ingredient per hectare to about 1,500 grams active ingredient per hectare, e.g., from about 50 grams to about 400 grams or from about 100 grams to about 350 grams. In some embodiments, the active ingredient is L-glufosinate. For example, the amount of L-glufosinate in the composition can be about 10 grams, about 50 grams, about 100 grams, about 150 grams, about 200 grams, about 250 grams, about 300 grams, about 350 grams, about 400 grams, about 500 grams, about 550 grams, about 600 grams, about 650 grams, about 700 grams, about 750 grams, about 800 grams, about 850 grams, about 900 grams, about 950 grams, about 1,000 grams, about 1,050 grams, about 1,100 grams, about 1,150 grams, about 1,200 grams, about 1,250 grams, about 1,300 grams, about 1,350 grams, about 1,400 grams, about 1,450 grams, or about 1,500 grams L-glufosinate per hectare.

The herbicidal compositions (including concentrates which require dilution prior to application to the plants) described herein contain L-glufosinate (i.e., the active ingredient), optionally some residual cyanhydrine or cyanhydrine derivative according to the formula (II), and one or more adjuvant components in liquid or solid form.

The compositions are prepared by admixing the active ingredient with one or more adjuvants, such as diluents, extenders, carriers, surfactants, organic solvents, humectants, or conditioning agents, to provide a composition in the form of a finely divided particulate solid, pellet, solution, dispersion, or emulsion. Thus, the active ingredient can be used with an adjuvant, such as a finely divided solid, a liquid of organic origin, water, a wetting agent, a dispersing agent, an emulsifying agent, or any suitable combination of these. From the viewpoint of economy and convenience, water is the preferred diluent. However, not all the compounds are resistant to hydrolysis and in some cases, this may dictate the use of non-aqueous solvent media, as understood by those of skill in the art.

Optionally, one or more additional components can be added to the composition to produce a formulated herbicidal composition. Such formulated compositions can include L-glufosinate, carriers (e.g., diluents and/or solvents), and other components. The formulated composition includes an effective amount of L-glufosinate.

A diluent can also be included in the formulated composition. Suitable diluents include water and other aqueous components. Optionally, the diluents are present in an amount necessary to produce compositions ready for packaging or for use.

The herbicidal compositions described herein, particularly liquids and soluble powders, can contain as further adjuvant components one or more surface-active agents in amounts sufficient to render a given composition readily dispersible in water or in oil. The incorporation of a surface-active agent into the compositions greatly enhances their efficacy. Surface-active agent, as used herein, includes wetting agents, dispersing agents, suspending agents, and emulsifying agents are included therein. Anionic, cationic, and non-ionic agents can be used with equal facility.

Suitable wetting agents include alkyl benzene and alkyl naphthalene sulfonates, sulfated fatty alcohols, amines or acid amides, long chain acid esters of sodium isothionate, esters of sodium sulfosuccinate, sulfated or sulfonated fatty acid esters petroleum sulfonates, sulfonated vegetable oils, ditertiary acetylenic glycols, polyoxyethylene derivatives of alkylphenols (particularly isooctylphenol and nonylphenol), and polyoxethylene derivatives of the mono-higher fatty acid esters of hexitol anhydrides (e.g. sorbitan). Exemplary dispersants include methyl cellulose, polyvinyl alcohol, sodium lignin sulfonates, polymeric alkyl naphthalene sulfonates, sodium naphthalene sulfonate, polymethylene bisnaphthalenesulfonate, and sodium N-methyl-N— (long chain acid) laurates.

Water-dispersible powder compositions can be made containing one or more active ingredients, an inert solid extender, and one or more wetting and dispersing agents. The inert solid extenders are usually of mineral origin, such as the natural clays, diatomaceous earth, and synthetic minerals derived from silica and the like. Examples of such extenders include kaolinites, attapulgite clay, and synthetic magnesium silicate. Water-dispersible powders described herein can optionally contain from about 5 to about 95 parts by weight of active ingredient (e.g., from about 15 to 30 parts by weight of active ingredient), from about 0.25 to 25 parts by weight of wetting agent, from about 0.25 to 25 parts by weight of dispersant, and from 4.5 to about 94.5 parts by weight of inert solid extender, all parts being by weight of the total composition. Where required, from about 0.1 to 2.0 parts by weight of the solid inert extender can be replaced by a corrosion inhibitor or anti-foaming agent or both.

Aqueous suspensions can be prepared by dissolution or by mixing together and grinding an aqueous slurry of a water-insoluble active ingredient in the presence of a dispersing agent to obtain a concentrated slurry of very finely divided particles. The resulting concentrated aqueous suspension is characterized by its extremely small particle size, so that when diluted and sprayed, coverage is very uniform.

Emulsifiable oils are usually solutions of active ingredient in water-immiscible or partially water-immiscible solvents together with a surface-active agent. Suitable solvents for the active ingredient described herein include hydrocarbons and water-immiscible ethers, esters, or ketones. The emulsifiable oil compositions generally contain from about 5 to 95 parts active ingredient, about 1 to 50 parts surface active agent, and about 4 to 94 parts solvent, all parts being by weight based on the total weight of emulsifiable oil.

Compositions described herein can also contain other additaments, for example, fertilizers, phytotoxicants and plant growth regulants, pesticides, and the like used as adjuvants or in combination with any of the above-described adjuvants. The compositions described herein can also be admixed with the other materials, e.g., fertilizers, other phytotoxicants, etc., and applied in a single application.

In each of the formulation types described herein, e.g., liquid and solid formulations, the concentration of the active ingredients are the same.

It is recognized that the herbicidal compositions can be used in combination with other herbicides. The herbicidal compositions of the present invention are often applied in conjunction with one or more other herbicides to control a wider variety of undesirable vegetation. When used in conjunction with other herbicides, the presently claimed compounds can be formulated with the other herbicide or herbicides, tank mixed with the other herbicide or herbicides or applied sequentially with the other herbicide or herbicides. Some of the herbicides that can be employed in conjunction with the compounds of the present invention include: amide herbicides such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flamprop and flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor and xylachlor; sulfonanilide herbicides such as benzofluor, perfluidone, pyrimisulfan and profluazol; sulfonamide herbicides such as asulam, carbasulam, fenasulam and oryzalin; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as benzfendizone, cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlomitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glyphosate and piperophos; phenoxy herbicides such as bromofenoxim, clomeprop, 2,4-DEB, 2,4-DEP, difenopenten, disul, erbon, etnipromid, fenteracol and trifopsime; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecoprop and mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazolyl herbicides such as benzofenap, pyrazolynate, pyrasulfotole, pyrazoxyfen, pyroxasulfone and topramezone; pyrazolylphenyl herbicides such as fluazolate and pyraflufen; pyridazine herbicides such as credazine, pyridafol and pyridate; pyridazinone herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluroxypyr, haloxydine, picloram, picolinafen, pyriclor, thiazopyr and triclopyr; pyrimidinediamine herbicides such as iprymidam and tioclorim; quaternary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vemolate; thiocarbonate herbicides such as dimexano, EXD and proxan; thiourea herbicides such as methiuron; triazine herbicides such as dipropetryn, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as butafenacil, bromacil, flupropacil, isocil, lenacil and terbacil; 3-phenyluracils; urea herbicides such as benzthiazuron, cumyluron, cycluron, dichloralurea, diflufenzopyr, isonoruron, isouron, methabenzthiazuron, monisouron and noruron; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluron and thidiazuron; and unclassified herbicides such as acrolein, allyl alcohol, aminocyclopyrachlor, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, flurochloridone, flurtamone, fluthiacet, indanofan, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan and tritac. The herbicidal compositions of the present invention can, further, be used in conjunction with glyphosate or 2,4-D on glyphosate-tolerant or 2,4-D-tolerant crops. It is generally preferred to use the compositions of the invention in combination with herbicides that are selective for the crop being treated and which complement the spectrum of weeds controlled by these compositions at the application rate employed. It is further generally preferred to apply the compositions of the invention and other complementary herbicides at the same time, either as a combination formulation or as a tank mix.

As mentioned above, the invention further relates in a third aspect to a method for selectively controlling weeds in an area, preferably containing a crop of planted seeds or crops that are resistant to glufosinate, comprising:
applying an effective amount of a composition comprising L-glufosinate and/or salts thereof obtained by the process of the present invention at an enantiomeric proportion of at least 50%, preferably in an enantiomeric excess of greater than 70%, over D-glufosinate and/or salts thereof and more than 0.01 wt.-% to less than 10 wt.-%, based on the total amount of the composition, of a cyanhydrine or cyanhydrine derivative according to the formula (II)

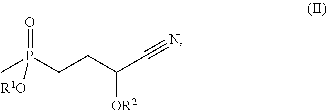

wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl, to the area.

In a preferred embodiment of the present invention, the composition comprises L-glufosinate and/or salts thereof at an enantiomeric proportion of 50 to 99%, preferably in an enantiomeric proportion of 60 to 98%, more preferably of 70 to 95%, and in particular of 80 to 90%, over D-glufosinate and/or salts thereof.

In a preferred embodiment of the present invention, the composition comprises 0.02 to 8 wt.-%, preferably 0.03 to 5 wt.-%, more preferably 0.05 to 3 wt.-%, and in particular 0.1 to 2 wt.-%, based on the total amount of the composition, of a cyanhydrine or cyanhydrine derivative according to the formula (II).

It is to be understood that the composition may comprise the same adjuvants and/or other herbicides as described in more detail above.

The compositions described herein are useful for application to a field of crop plants for the prevention or control of weeds. The composition may be formulated as a liquid for spraying on a field. The L-glufosinate is provided in the composition in effective amounts. As used herein, effective amount means from about 10 grams active ingredient per hectare to about 1,500 grams active ingredient per hectare, e.g., from about 50 grams to about 400 grams or from about 100 grams to about 350 grams. In some embodiments, the active ingredient is L-glufosinate. For example, the amount of L-glufosinate in the composition can be about 10 grams, about 50 grams, about 100 grams, about 150 grams, about 200 grams, about 250 grams, about 300 grams, about 350 grams, about 400 grams, about 500 grams, about 550 grams, about 600 grams, about 650 grams, about 700 grams, about 750 grams, about 800 grams, about 850 grams, about 900 grams, about 950 grams, about 1,000 grams, about 1,050 grams, about 1,100 grams, about 1,150 grams, about 1,200 grams, about 1,250 grams, about 1,300 grams, about 1,350 grams, about 1,400 grams, about 1,450 grams, or about 1,500 grams L-glufosinate per hectare.

The present invention is further illustrated by the following examples.

### EXAMPLES

### Preparation of Enzymes

### a) Cloning of Enzyme Genes (Ex 1)

The amino acid sequences of the respective enzymes were identified from public databases (UniProt, https://www.uniprot.org; NCBI protein database, https://www.ncbi.nlm.nih.gov/protein. Sequences from NCBI are indicated by an “*” at the beginning of the respective database identifier). The respective DNA sequence was derived thereof using standard codon usage of Escherichia coli. The DNA sequence was synthesized (BioCat GmbH) and cloned into the plasmid pDHE19.2 (Ress-Loeschke, M. et al., DE 19848129, 1998, (BASF AG)). The resulting plasmids were used to transform competent cells (Chung, C. T. et al., Proc Natl Acad Sci USA, 1989, 86, 2172) of the E. coli strain TG10, pAgro, pHSG575 (E. coli TG10 (Kesseler, M. et al., WO2004050877A1, 2004, (BASF AG)):rhaA−—derivate of E. coli TG1 transformed with pHSG575 (Takeshita, S. et al., Gene, 1987, 61, 63) and pAgro4 (pBB541 in Tomoyasu, T. et al., Mol. Microbiol., 2001, 40, 397).

### b) Recombinant Production of Enzymes (Ex 2)

### Biocatalyst Preparation in Shake Flasks

E. coli TG10 carrying the recombinant plasmid of the enzyme was used to inoculate 2 ml LB medium (Bertani, G., J Bacteriol, 1951, 62, 293) supplemented with 100 μg/ml ampicillin, 100 μg/ml spectinomycin, 20 μg/ml chloramphenicol and the resulting pre-culture was incubated for 5 h at 37° C. at an agitation of 250 rpm. 1 ml of the pre-culture was used to inoculate 100 ml LB medium supplemented with 100 μg/ml ampicillin, 100 μg/ml spectinomycin, 20 μg/ml chloramphenicol, 1 mM MnCl2, 0.1 mM isopropyl-β-D-thiogalactopyranosid, and 0.5 g/l rhamnose in a 500 ml baffled Erlenmeyer-flask. The culture was incubated at 37° C. for 18 h under shaking conditions. Subsequently, the biomass was harvested by centrifugation at 3220×g for 10 min at 8° C. The supernatant was discarded, and the cell pellet resuspended in 8 ml HEPES buffer at a concentration of 100 mM and pH 8.2 supplemented with 1 mM MnCl2. The cell suspension was used without any further preparation for synthesis in case whole cell biotransformation were carried out. In case cleared cell lysates were employed instead, 5 ml of the cell suspension were distributed into 5 reaction tubes containing lysing matrix B (0.7 ml quartz-beads at Ø 0.1 mm, MP Biomedicals), the tubes chilled on ice, and cells subsequently broken in a homogenizer (Peqlab Precellys24, VWR) for two 30 second cycles. In between cycles samples were chilled on ice. The resulting cell free lysates were cleared by centrifugation 20817 xg for 10 min, at 8° C. The supernatants were isolated and fractions from the same batch combined (=cleared cell lysate).

### Fermentative Whole-Cell Biocatalyst Production

E. coli TG10 containing the plasmids pAgro4 and pHSG575 were transformed with pDHE plasmid encoding the protein of interest. Transformants were cultivated on a LB agar plate supplemented with 100 μg/ml ampicilin, 100 μg/ml spectinomycin, and 20 μg/ml chloramphenicol.

Preculture medium:
EcoK12 solution

| | | |
|---|---|---|
| Ultrapure water | 1.0 | kg |
| Citric acid monohydrate | 40.0 | g |
| Zinc sulfate heptahydrate | 11.0 | g |
| Diammonium iron sulfate hexahydrate | 8.6 | g |
| Manganese sulfate monohydrate | 3.0 | g |
| Copper sulfate pentahydrate | 0.8 | g |
| Cobalt sulfate heptahydrate | 0.09 | g |

Sterilized by filtration using a filter with 0.2 μm pore size.

Part 1

| | | |
|---|---|---|
| Ultrapure water | 1.0 | kg |
| Citric acid monohydrate | 3.4 | g |
| Magnesium sulfate heptahydrate | 2.4 | g |
| Calcium chloride dihydrate | 0.1 | g |
| EcoK12 solution | 20 | g |

| | |
|---|---|
| Sodium hydroxide solution 25% | used to adjust pH to 6.6 |

Part 2

| | | |
|---|---|---|
| Ultrapure water | 500 | g |
| Potassium dihydrogen phosphate | 26.6 | g |
| Diammonium hydrogen phosphate | 8.0 | g |

| | |
|---|---|
| Sodium hydroxide solution 25% | used to adjust pH to 6.4 |

Part 3

| | | |
|---|---|---|
| Ultrapure water | 500 | g |
| Glycerol 99% | 36.0 | g |
| Sodium gluconate | 24.0 | g |

| | |
|---|---|
| Phosphoric acid 20% | used to adjust pH 6.6 |

All 3 parts were sterilized at 121° C. for 30 minutes.

Vitamin solution

| | | |
|---|---|---|
| Ultrapure water | 100 | g |
| Thiamine hydrochloride | 1.0 | g |
| Vitamin B₁₂ | 0.5 | g |

Sterilized by Filtration Using a Filter with 0.2 μm Pore Size

To make up the final preculture medium parts 1, 2, and 3 are combined and 2.0 ml of vitamin solution added. Furthermore, the medium was supplemented with 100 μg/ml ampicillin, 100 μg/ml spectinomycin, and 20 μg/ml chloramphenicol. Several transformants were scraped of the LB agar plate and used to inoculated 2×100 g of preculture media in 1|baffled Erlenmeyer flasks. These precultures were incubated at 37° C. and 150 rpm. When an OD600 of 12 was reached the precultures were used in their entirety to inoculate the main culture.

Main culture medium:
Part 4

| Ultrapure water | 9.6 | kg |
|---|---|---|
| Citric acid monohydrate | 21.1 | g |
| Potassium dihyrodgen phosphate | 173.6 | g |
| Diammonium hydrogen phosphate | 52.8 | g |
| Mangesium sulfate heptahydrate | 15.1 | g |
| Calcium chloride dihydrate | 0.7 | g |
| EcoK12 solution | 123 | g |
| Sodium hydroxide solution 25% | adjusted pH to 6.4 | |
| Pluriol P 2000 | 1 | ml |

Part 4 was sterilized at 125° C. for 45 min.

Part 5

| Ultrapure water | 300 | g |
|---|---|---|
| Thiamine hydrochloride | 151 | mg |
| Vitamin B12 | 30.2 | mg |
| Ampicillin sodium salt | 1000 | mg |
| Spectinomycin hydrochloride | 500 | mg |
| Chloramphenicol | 200 | mg |
| Part 5 was sterilized by sterile filtration | | |
| using a filter unit with a pore size of 0.1 μm | | |
| Glycerol solution | | |
| Ultrapure water | 804 | g |
| Citric acid monohydrate | 29.1 | g |
| Sodium sulfate | 58.1 | g |
| Diammonium iron sulfate hexahydrate | 4.5 | g |
| Glycerol 99% | 3370 | g |

Thiamine solution

| | | |
|---|---|---|
| Ultrapure water | 40 | g |
| Thiamine hydrochloride | 55 | mg |

Antifoam solution

| | | |
|---|---|---|
| Pluriol P 2000 | 350 | g |

Base solution

| | | |
|---|---|---|
| Ammonia water 25% | 1500 | ml |

Inductor solution

| | | |
|---|---|---|
| Ultrapure water | 150 | g |
| Rhamnose monohydrate | 100 | g |
| IPTG | 238 | mg |

Glycerol, and antifoam solution were sterilized at 121° C. for 30 min. Thiamine and inductor solution are sterilized by filtration using a filter with a pore size of 0.2 μm.

Parts 4 and 5 were combined in the sterilized fermentation vessel (Techfors, Infors HT) and inoculated with the preculture. The vessel was kept at a temperature of 37° C., a pressure of 0.2 bar, and at a pH of 6.6 by dosing with base solution over the course of fermentation. The pO2 level was kept at 20-40% by adjusting the stirrer speed (commonly 500 rpm) and aeration rate (commonly 6 l/min). Antifoam solution was added as needed. Glycerol and thiamine solutions were combined yielding the feed solution. After inoculation the feed solution was dosed at a rate of 10 g/h. After 7 h the dosing of the feed solution was switched to “stop and see” mode in which feed was activated at a rate of 10 g/h upon increase of pO2-level. After 14 h or 330 g of feed solution consumption the feed rate was increased to 80-100 g/h. Gene expression was induced at an oxygen transfer rate of 80 mmol/l/h or alternatively at an OD600 of 12 by addition of inductor solution. The fermentation was stopped 36 h post induction by lowering the temperature to 15° C. The cooled fermentation broth was drained from the fermenter and centrifuged at 4700 rpm and 10° C. to pellet the cells. The resulting supernatant was discarded, and cells resuspended in 3850 g of 50 mM potassium dihydrogen phosphate buffer at pH 7.0. The cell suspension was frozen at −80° C. before being lyophilized. In that regard, the lyophilizer was kept at −50° C. and a pressure of 0.25 mbar. Lyophilized cells were stored at 4° C.

### Production of Lyophilized Cell Free Extracts

Lyophilized cells were resuspended in ultrapure water at 100 g/l. The cell suspension was cooled on ice before cells were disrupted by three passages through a pressure homogenizer (Panda Plus 2000, GEA) which was set to 800 bar. Pressures of the three passages were commonly between 1000 to 1400 bar. The resulting mixture was cleared from debris by centrifugation at 10000 rpm at 10° C. for 15 min. The resulting pellet was discarded and the concentration of protein in the supernatant analyzed by Bradford assay. The supernatant was frozen at −80° C. and subsequently lyophilized at −50° C. and a pressure of 0.25 mbar.

### Preparation of Starting Materials

### c) Synthesis of n-Butyl (3-cyano-3-hydroxypropyl)methylphosphinat (ACM-H) (Ex 3)

ACM-H has been prepared according to example 2 of WO 2015/173146 A1.

### d) Synthesis of n-Butyl (3-cyano-3-acetoxypropyl)methylphosphinat (ACM) (Ex 4)

ACM has been prepared according to example 1 of WO 2017/037012 A1.

### Preparation of L-Glufosinate P-Butyl Ester

### e) Preparation of L-glufosinate butyl ester from ACM-H (IE 5)

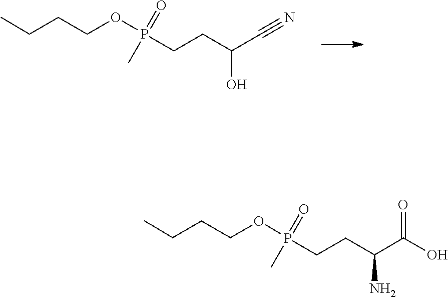

2.47 g of ammonium hydrogen carbonate (31.2 mmol, 1.7 equiv.) were dissolved under stirring in 50 ml distilled water. 4 g of n-Butyl (3-cyano-3-hydroxypropyl)methylphosphinat (18.2 mmol, 1 equiv. “ACM-H”) prepared according to Ex 3 were added under stirring. The resulting solution was stirred at 37° C. 1 ml of a 2 M aqueous MnCl₂ solution were added followed by enzymes (Uniprot ID: A0A159Z531_9RHOB, SEQ ID NO:1, 1 g lyophilized cell-free extract and A0A535Y1H2_UNCCH, SEQ ID NO: 2, 500 mg, lyophilized cell free extract) to yield a 357 mM solution of “ACM-H”. The solution was stirred at 37° C.

After 3.5 h of stirring, further 0.5 g of enzyme (A0A535Y1H2_UNCCH, SEQ ID NO: 2, 500 mg, lyophilized cell free extract) were added. After 27 h of stirring 2.47 g of ammonium hydrogen carbonate (31.2 mmol, 1.7 equiv.) were added in combination with enzymes (Uniprot ID: A0A159Z531_9RHOB, SEQ ID NO:1, 1 g lyophilized cell-free extract and A0A535Y1H2_UNCCH, SEQ ID NO: 2, 500 mg, lyophilized cell free extract). After a total reaction time of 99 h additional enzyme ((A0A535Y1H2_UNCCH, SEQ ID NO: 2, 500 mg, lyophilized cell free extract) was added and after 116 h the reaction was stopped.

The final concentration of Butyl-glufosinate was 56 mmol as measured by HPLC, which corresponds to a conversion of 15 mol % of ACM-H. The enantiomeric ratio was 92% L:8% D. After the reaction had finished, the crude reaction mixture was heated to 80° C. for 30 min and filtered to remove the cell lysate. The filtrate was concentrated under reduced pressure. L-glufosinate butyl ester was separated on a Dowex-50 WX 8 200-400 (H) eluting with ammonia (1 M in water). The concentrations of the butyl esters of L and D-Glufosinate were determined by HPLC-MS using a Supelco Chirobiotic T2 (Gradient 90% ACN/Water to 60% ACN/Water in 19 minutes, 0.1% Formic acid). Temp: 20° C., flow rate 0.8 mL/min. Retention times of Butyl ester of Glufosinate: L-configured Diastereoisomers (13.3+13.7 min); D-configured (14.5 and 16.8 min).

### f) Preparation of L-Glufosinate Butyl Ester from ACM (IE 6)

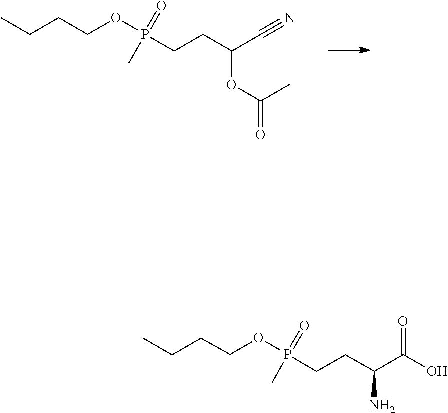 [SMILES: CCCCOP(C)(=O)CCC(C#N)OC(C)=O ; CCCCOP(C)(=O)CC[C@H](N)C(=O)O]

Diammonium carbonate (9.6 g) was dissolved in water (100 mL) and the pH was adjusted with HCl (37% in water) to 8.5. 20 ml of the resulting buffer were used to dissolve 3.25 g of n-Butyl (3-cyano-3-acetoxypropyl)methylphosphinat (11.2 mmol, 1 equiv. “ACM”, 90%) prepared according to Ex 4. The resulting reaction mixture was stirred at 30° C. 0.5 ml of a 2 M aqueous MnCl₂ solution were added followed by enzymes (Uniprot ID: A0A159Z531_9RHOB, SEQ ID NO:1, 2 g lyophilized cell-free extract and A0A535Y1H2_UNCCH, SEQ ID NO: 2, 1 g, lyophilized cell free extract) to yield a 546 mM solution of “ACM”. The solution was stirred at 30° C. for 94 h.

After this time the final concentration of Butyl-glufosinate was 55 mmol as measured by HPLC, which corresponds to a conversion of 10 mol % of ACM. The enantiomeric ratio was >99% L:<1% D.

### g) Preparation of L-Glufosinate from ACM Using a Heating Step (IE 6)

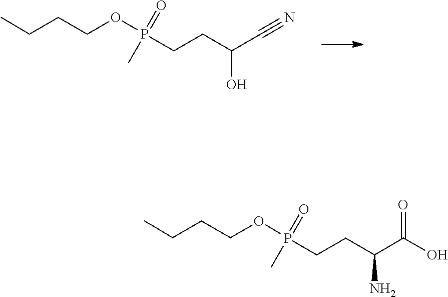

2.16 g of ammonium hydrogen carbonate were dissolved under stirring in 12.5 ml distilled water. 2 g of n-Butyl (3-cyano-3-hydroxypropyl)methylphosphinat (“ACM-H”) prepared according to Ex 3 were added under stirring. The resulting solution was stirred at 80° C. under microwave heating for 1 h. Subsequently, the solution was allowed to cool to 37° C. temperature and 250 μl of a 2 M aqueous MnCl₂ solution were added followed by addition of the two amidases (Uniprot ID: A0A159Z531_9RHOB, SEQ ID NO:1, 500 mg lyophilized cell-free extract) and (A0A535Y1H2_UNCCH, SEQ ID NO: 2, 250 mg, lyophilized cell free extract). The solution was agitated further at 37° C. After 4.5 h amidase (A0A535Y1H2_UNCCH, SEQ ID NO: 2, 250 mg, lyophilized cell free extract) was added. After 46 h the pH was readjusted to 8.5 and the two amidases were added again (Uniprot ID: A0A159Z531_9RHOB, SEQ ID NO:1, 500 mg lyophilized cell-free extract) and (A0A535Y1H2_UNCCH, SEQ ID NO: 2, 250 mg, lyophilized cell free extract). After 52 h HPLC showed a conversion to Butyl-Glufosinate of 15 mol %. The enantiomeric ratio was 95% L:5% D. After the reaction had finished, the crude reaction mixture was heated to 80° C. for 30 min and filtered to remove the cell lysate and the filtrate concentrated under reduced pressure. L-glufosinate butyl ester was separated on a Dowex-50 WX 8 200-400 (H) eluting with ammonia (1 M in water) and further subjected to reverse phase chromatography (Gradient of Acetonitrile in water with 0.1% trifluoroacetic acid). An analytical sample of L-glufosinate butyl ester (50 mg) was stirred with HCl in water (18% wt) at 100° C. for 5 h. The enantiomeric ratio was determined by chiral HPLC (92% L-Glufosinate: 8% D-Glufosinate).

### h) Preparation of L-Glufosinate Butyl Ester from ACM Using a Heating Step (IE 7)

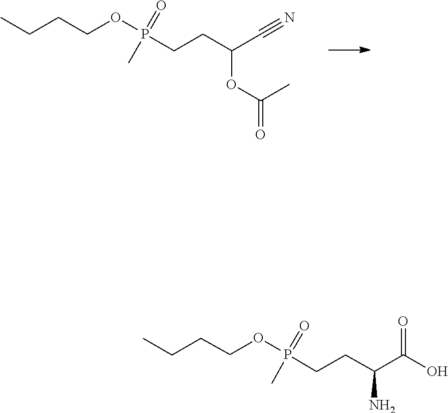 [SMILES: CCCCOP(C)(=O)CCC(C#N)OC(C)=O ; CCCCOP(C)(=O)CC[C@H](N)C(=O)O]

1.6 g of ammonium hydrogen carbonate were dissolved under stirring in 12.5 ml distilled water. 2 g of n-Butyl (3-cyano-3-acetoxypropyl)methylphosphinat (“ACM”, 90%) prepared according to Ex 4 were added under stirring. The resulting solution was stirred at 80° C. under microwave heating for 3 h. Subsequently, the solution was allowed to cool to 40° C. temperature and the pH was adjusted with aqueous ammonia to 8.5. 375 μl of a 2 M aqueous MnCl₂ solution were added followed by addition of the two amidases (Uniprot ID: A0A159Z531_9RHOB, SEQ ID NO:1, 500 mg lyophilized cell-free extract) and (A0A535Y1H2_UNCCH, SEQ ID NO: 2, 250 mg, lyophilized cell free extract). The solution was agitated at 40° C. for 93 h. After this time HPLC showed that 22 mol % of ACM had converted to Butyl-Glufosinate. The enantiomeric ratio was determined by chiral HPLC (>99% L<1% D).

(from Defluviimonas alba)
SEQ ID NO: 1
MTLIVTNGRVVSPEGVALRDVVVEGETIAAVLPAGEAVKACPGAE
VIDATGRIVIPGGVDPHVHLLVGFMGQRSVYDFASGGIAALRGGT
TAIVDFALQRRGGSMLKGLAHRRKQADANVTLDYGLHLIVTDVTA
DTLAELPALRAAGVTTLKVYTVYEEDGLKVEDGALFALMQGAARH
GLSVVLHAENAGIVERLRAEAVARGDTHPRHHALTRPPIVEIEAV
SRAIAFSRATGCGVHILHLVAADAIALVAAARAEGLPVTAETCSH
YLALTDEALERPNGHEFILSPPLRDKANQDRLWKGLETAALSLVA
SDEVSYSAAAKAMGLPSFATVANGITGIEARLPLLYTLGVDQGRI
GLQRFVKLFSTWPAEIFGFAGKGRIAPGFDADLVLIDPDGRRVIS
TDSDYGDIGYTPYAGMELTGFATETIYRGRLVVRDGVFLGTEGQG
RFIERVAPRRPAP
(from Chloroflexi bacterium)
SEQ ID NO: 2
MTDAARLERRIHELAQIGRTDDPAREIYATAVSRLGLSAEEQRAR
DLVTSWCAPHGATARRDPAANLYLRFPGADPHAPVVLVGSHLDSV
PMGGRFDGALGVCCAVEAVVSLLESGARFARPVEVVGWADEEGAR
FGYGLFGSAAAFGRLRVDPERVRDKGGTSIAEALRALGESGDLAG
AMRDPKGIRAYLELHIEQGPRLERAGAPLGVVSDIVGIFHGLVMV
RGEQNHAGATVMGERHDALVAASHMIIALERIASSVPDAVATVGE
ITVKPGAKNVIPGECTFSLDIRAPKQESIDLVLERFKAEANEIFR
KSLREWGLRPLQSVAVTPLDEDLRDLLWKSAMSVGVNAPTLVSGA
GHDAQNPSLAGVPTGMIFVRSTGGSHTPTEFAATADAALGAKALE
IAIRELATA

1. A process for preparing L-glufosinate and/or a salt thereof or an L-glufosinate alkyl ester and/or a salt thereof, wherein the L-glufosinate or the L-glufosinate alkyl ester have a molecular structure according to formula (I): 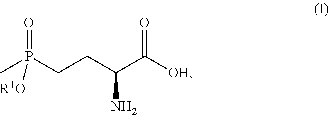
wherein R¹ is H or C₁-C₈ alkyl,
wherein the process comprises the step of reacting the following components in at least one reaction step:
(1) a cyanhydrine or cyanhydrine derivative according to formula (II) 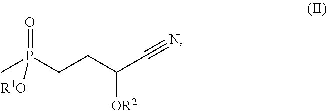
wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl;
(2) a source of ammonia;
(3) a source of carbon dioxide; and
(4) at least two enzymes.

2. The process according to claim 1, wherein the component (4) comprises, preferably consists of (4a) at least one amidohydrolase acting on cyclic amides (EC 3.5.2) and (4b) at least one L-Amidohydrolase acting on linear amides (EC 3.5.1).

3. The process according to claim 1 or 2, wherein initially the components (1), (2), and (3) are contacted, subsequently component (4) is added, preferably first component (4a) and finally component (4b) are added.

4. The process according to claims 1 to 3, wherein the components (1) to (4), preferably (1) to (4b), are added in the same reaction step, preferably wherein the reaction is carried out as a one pot reaction.

5. The process according to any of the preceding claims, wherein the cyanhydrine is prepared by the reaction of an aldehyde and cyanide, preferably hydrogen cyanide or potassium cyanide, and wherein the aldehyde has a molecular structure according to formula (III): 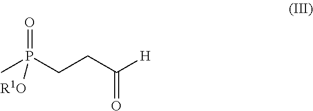
wherein R¹ is H or C₁-C₈ alkyl, preferably H or C₁-C₆ alkyl, more preferably H or C₂-C₄ alkyl, even more preferably H, ethyl or butyl, and most preferably ethyl.

6. The process according to any of the preceding claims, wherein R¹ is C₁-C₈ alkyl, preferably C₁-C₆ alkyl, more preferably C₂-C₄ alkyl, even more preferably ethyl or butyl, and most preferably ethyl.

7. The process according to any of the preceding claims, wherein the source of ammonia is selected from the list consisting of gaseous ammonia, solubilized ammonia, an ammonium salt, or mixtures thereof.

8. The process according to any of the preceding claims, wherein the source of carbon dioxide is gaseous carbon dioxide, solubilized carbon dioxide, a carbonate salt, or mixtures thereof.

9. The process according to any of the preceding claims, wherein the cyanhydrine or cyanhydrine derivative is a cyanhydrine derivative according to formula (IV): 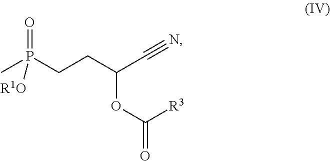
wherein R³ is C₁-C₈ alkyl, preferably C₁-C₄ alkyl, more preferably C₁-C₃ alkyl, and most preferably methyl.

10. The process according to any of the preceding claims, wherein the L-glufosinate and/or the salt thereof or the L-glufosinate alkyl ester and/or the salt thereof are prepared in enantiomeric excess, preferably in an enantiomeric excess of more than 85%, more preferably more than 90%, even more preferably more than 95%, and most preferably more than 99%.

11. The process according to any of the preceding claims, wherein the amidohydrolase acting on cyclic amides (EC 3.5.2) is an L-amidohydrolase acting on cyclic amides (EC 3.5.2).

12. The process according to any of the preceding claims, wherein the L-amidohydrolase acting on cyclic amides (EC 3.5.2) is selected from the group of enzymes identified by their Uniprot ID or NCBI ID (the latter being indicated by an “*” at the beginning of the ID) consisting of O69809 and variants thereof, Q846U5_9BACL and variants thereof, P81006 and variants thereof, Q84FR6_9MICC and variants thereof, Q56S49_9BACI and variants thereof, A1E351_9BACI and variants thereof, Q28SA7 and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B5L363 and variants thereof, P42084 and variants thereof, P25995 and variants thereof, Q3Z354 and variants thereof, B1XEG2 and variants thereof, Q9F465_PAEAU and variants thereof, A0A161KD37_9CHLR and variants thereof, A0A1J4XHR4_9BACT and variants thereof, A0A1C4QIY5_9ACTN and variants thereof, A0A0K2UMP4_LEPSM and variants thereof, A0A159Z531_9RHOB and variants thereof, E1R8C9_SEDSS and variants thereof, A0A1F9 QT17_9BACT and variants thereof, A0A0D8IVV8_9FIRM and variants thereof, A0A0B5QKE4_CLOBE and variants thereof, A0A0N1GBZ8_9ACTN and variants thereof, A0A174ADZ3_9FIRM and variants thereof, U7V9Q6_9FUSO and variants thereof, A0A0J1FAI4_9FIRM and variants thereof, PHYDA_ECOK1 and variants thereof, A0A0S8H576_9BACT and variants thereof, A0A1J4J4Y8_9EUKA and variants thereof, A0A0D5NFS5_9BACL and variants thereof, A0A0D5NNJ7_9BACL and variants thereof, A0A1H2AV66_9BACL and variants thereof, A0A0Q4RXY0_9BACL and variants thereof, A0A0Q7SB75_9BACL and variants thereof, A0A100VRN2_PAEAM and variants thereof, W4BDJ0_9BACL and variants thereof, A0A1J5E082_9DELT and variants thereof, A0A1H5ZFN3_9BACT and variants thereof, A0A1F8NMM2_9CHLR and variants thereof, A0A1F8SDV1_9CHLR and variants thereof, A0A1H1PLX0_9BACT and variants thereof, A0A0Q518X4_9DEIO and variants thereof, *WP_046170519.1 and variants thereof, *WP_023514195.1 and variants thereof, *WP_023516147.1 and variants thereof, and *ANZ15483.1, wherein variants are defined as polypeptide sequences with at least 80%, preferably 90%, and most preferably 95%, sequence identity to the respective polypeptide sequence, preferably are selected from the group of enzymes identified by their Uniprot ID or NCBI ID (the latter being indicated by an “*” at the beginning of the ID) consisting of Q45515 and variants thereof, Q44184 and variants thereof, P81006 and variants thereof, A0A1C4QIY5_9ACTN and variants thereof, A0A0K2UMP4_LEPSM and variants thereof, *WP_046170519.1 and variants thereof, A0A159Z531_9RHOB and variants thereof, and E1R8C9_SEDSS, and variants thereof, wherein variants are defined as polypeptide sequences with at least 80%, preferably 90%, and most preferably 95%, sequence identity to the respective polypeptide sequence.

13. The process according to any of the preceding claims, wherein the L-Amidohydrolase acting on linear amides (EC 3.5.1) is selected from the group of enzymes identified by their Uniprot ID consisting of A0A0K9YX84_9BACL and variants thereof, E3HUL6_ACHXA and variants thereof, A0A4D7Q548_GEOKU and variants thereof, Q9F464 and variants thereof, A0A2S9D976_9MICC and variants thereof, A0A3E0C996_9BURK and variants thereof, A0A535Y1H2_UNCCH and variants thereof, A0A6P2ISL4_BURL3 and variants thereof, A0A1Y4GC62_9BACT and variants thereof, wherein variants are defined as polypeptide sequences with at least 80%, preferably 90%, and most preferably 95%, sequence identity to the respective polypeptide sequence, preferably are selected from the group of enzymes identified by their Uniprot ID consisting of A0A3E0C996_9BURK and variants thereof, A0A535Y1H2_UNCCH and variants thereof, A0A6P2ISL4_BURL3 and variants thereof, A0A1Y4GC62_9BACT wherein variants are defined as polypeptide sequences with at least 80%, preferably 90%, and most preferably 95%, sequence identity to the respective polypeptide sequence.

14. A composition comprising a cyanhydrine or cyanhydrine derivative according to formula (II): 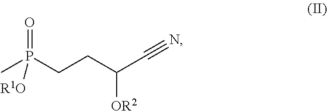
wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl, and L-glufosinate and/or salts thereof.

15. A method for selectively controlling weeds in an area, preferably containing a crop of planted seeds or crops that are resistant to glufosinate, comprising:
applying an effective amount of a composition comprising L-glufosinate and/or salts thereof at an enantiomeric proportion of at least 50%, preferably in an enantiomeric excess of greater than 70%, over D-glufosinate and/or salts thereof and more than 0.01 wt.-% to less than 10 wt.-%, based on the total amount of the composition, of a cyanhydrine or cyanhydrine derivative according to formula (II): 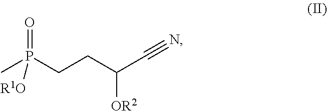
wherein
R¹ is H or C₁-C₈ alkyl, and
R² is H, C₁-C₈ alkyl, C₆-C₁₀ aryl, C₇-C₁₀ aralkyl, C₄-C₁₀ cycloalkyl, or C₁-C₁₀ acyl, to the area.


SEQUENCE LISTING (2 sequences)

SEQ ID NO: 1 - protein, 463 aa, Defluviimonas alba
MTLIVTNGRVVSPEGVALRDVVVEGETIAAVLPAGEAVKACPGAEVIDATGRIVIPGGVDPHVHLLVGFMGQRSVYDFASGGIAALRGGTTAIVDFALQRRGGSMLKGLAHRRKQADANVTLDYGLHLIVTDVTADTLAELPALRAAGVTTLKVYTVYEEDGLKVEDGALFALMQGAARHGLSVVLHAENAGIVERLRAEAVARGDTHPRHHALTRPPIVEIEAVSRAIAFSRATGCGVHILHLVAADAIALVAAARAEGLPVTAETCSHYLALTDEALERPNGHEFILSPPLRDKANQDRLWKGLETAALSLVASDEVSYSAAAKAMGLPSFATVANGITGIEARLPLLYTLGVDQGRIGLQRFVKLFSTWPAEIFGFAGKGRIAPGFDADLVLIDPDGRRVISTDSDYGDIGYTPYAGMELTGFATETIYRGRLVVRDGVFLGTEGQGRFIERVAPRRPAP

SEQ ID NO: 2 - protein, 414 aa, Chloroflexi bacterium
MTDAARLERRIHELAQIGRTDDPAREIYATAVSRLGLSAEEQRARDLVTSWCAPHGATARRDPAANLYLRFPGADPHAPVVLVGSHLDSVPMGGRFDGALGVCCAVEAVVSLLESGARFARPVEVVGWADEEGARFGYGLFGSAAAFGRLRVDPERVRDKGGTSIAEALRALGESGDLAGAMRDPKGIRAYLELHIEQGPRLERAGAPLGVVSDIVGIFHGLVMVRGEQNHAGATVMGERHDALVAASHMIIALERIASSVPDAVATVGEITVKPGAKNVIPGECTFSLDIRAPKQESIDLVLERFKAEANEIFRKSLREWGLRPLQSVAVTPLDEDLRDLLWKSAMSVGVNAPTLVSGAGHDAQNPSLAGVPTGMIFVRSTGGSHTPTEFAATADAALGAKALEIAIRELATA